(19) United States
(12) Patent Application Publication
(10) Pub. No.: US 2021/0139882 A1
(43) Pub. Date: May 13, 2021

(54) MICROCAPSULE OF SUSTAINABLE SELF-HEALING COAL MINE VENTILATION SEALING MATERIAL CRACK AND PREPARATION METHOD THEREOF

(71) Applicant: SHANDONG UNIVERSITY OF SCIENCE AND TECHNOLOGY, Qingdao City (CN)

(72) Inventors: Xiangming HU, Qingdao City (CN); Weimin CHENG, Qingdao City (CN); Mingyue WU, Qingdao City (CN); Yanyun ZHAO, Qingdao City (CN); Di XUE, Qingdao City (CN)

(21) Appl. No.: 17/257,156

(22) PCT Filed: Aug. 14, 2018
(86) PCT No.: PCT/CN2018/100497

§ 371 Date: Dec. 30, 2020

(30) Foreign Application Priority Data
Jul. 20, 2018 (CN) 201810799798.9

(51) Int. Cl. C12N 11/14; C04B 20/10; C04B 18/02

(52) U.S. Cl. CPC C12N 11/14; C04B 2103/0001; C04B 18/021; C04B 20/1085

(57) ABSTRACT

A microcapsule of sustainable self-healing coal mine ventilation sealing material crack. The microcapsule includes a microcapsule core material and a microcapsule wall material. The microcapsule core material is prepared using a bacterial lyophilized powder and a substrate. By using urease-producing bacteria, there is provided a method of protecting bacteria to survive a long time in the cement-based material, supplying sufficient nutrient substances and reducing the impact of the doping of bacteria on the mechanical property of the cement-based material. The bacterial lyophilized powder and substrate are prepared into microcapsules which are added into the cement-based material when the cement-based material is mixed. In this case, once concrete cracks, the microcapsules breaks and the spores in the material are activated to perform normal metabolism so as to induce precipitation of calcium carbonate continuously, thereby continuously realizing self-healing of coal mine ventilation sealing material cracks.

### TECHNICAL FIELD

The present disclosure relates to the technical field of coal mine ventilation sealing material crack healing and in particular to a microcapsule of sustainable self-healing coal mine ventilation sealing material crack and a preparation method thereof.

### BACKGROUND

Along with application of pillar-less techniques of gob-side entry retaining and gob-side entry driving and the like in the reform of coal mining technologies, the possibility of ventilation of the coal roadways is increased noticeably, bringing great difficulties to management of coal seams liable to spontaneous combustion. Therefore, the ventilation sealing work of coal roadways is very important. At present, some common spraying materials for sealing ventilation are cement type material, coal powder ash foam material, polymer spraying material, colloid, foam material and polymer elastomer, which are applied to some degree in different mine districts. Such materials play important role in guaranteeing safe production of coal mines. Due to its high strength and low cost and so on, the cement-based material is widely applied in the coal mines. However, along with increasing coal mining depth, the stress of surrounding rocks increases, and the cement-based material will inevitably produce a large quantity of cracks under the strata pressure. If such cracks cannot be found and repaired in time, ventilation channels will be formed, which lead to spontaneous combustion of gob and gas emission. Therefore, it is extremely important to find and repair such cracks in time.

At present, the common crack repairing method includes a surface sealing method, a crack sealing method, and a structural strengthening and reinforcement method and so on. But the surface sealing method cannot penetrate into the interior of the cracks and thus is not applicable to cracks with a water pressure. The materials commonly used for grouting and caulking in the crack sealing method are organic materials such as epoxy resin, and polyurethane, which have a thermal expansion coefficient different from that of the cement-based material and have a poor compatibility. In addition, the organic chemical materials are mostly volatile and release gases harmful to human and environment. The structural strengthening and reinforcement method features complex operation and high cost. Thus, a new crack repairing method is urgently needed.

Microbially induced calcium carbonate precipitation (MICP), as a new technique, is applied in concrete repair. The microbially induced precipitation is considered as a good crack repairing method due to its good compatibility with cement-based materials, low price, simple operation and no pollution. During the mixing process of the cement-based material, the method adds urease-producing bacteria and its substrate into the cement-based material. Due to high alkalinity, high Ca²⁺ concentration, lack of nutrient substances in the cement-based material and pore shrinkage in hydration and so on, bio-mineralization cannot be better applied in repairing the cracks of the cement-based material. Therefore, it is necessary to provide a method of not only protecting bacteria to survive a long time in the cement-based material but also improving crack repairing efficiency.

### SUMMARY

In order to solve the technical problem of the above coal mine ventilation sealing material crack repairing, the present disclosure provides a microcapsule of sustainable self-healing coal mine ventilation sealing material crack and a preparation method thereof.

In order to achieve the above purpose, the present disclosure adopts the following technical solution.

A microcapsule of sustainable self-healing coal mine ventilation sealing material crack includes a microcapsule core material, and a microcapsule wall material, and the microcapsule core material is prepared using a bacterial lyophilized powder and a substrate.

Preferably, a mass ratio of the microcapsule core material to the microcapsule wall material is 1:1.2-1.5.

Preferably, a granule size of the microcapsule core material is 1.9-2.5 mm with a wall thickness being 60-100 μm.

Preferably, the bacterial lyophilized powder in the microcapsule core material is selected from one or more of bacillus sphaericus, sporosarcina pasteurii and bacillus cereus.

Preferably, the substrate in the microcapsule core material is urea, soluble calcium salt, nutrient substance and thickener; the soluble calcium salt is selected from one or more of calcium chloride, calcium lactate, calcium nitrate, calcium formate and calcium acetate, the nutrient substance is selected from one or more of inosine, peptone and yeast powder; and the thickener is selected from one or more of microcrystalline cellulose, xanthan gum, and hydroxy propyl methyl cellulose.

Preferably, the mass ratio of the bacterial lyophilized powder to the substrate is 1:50-70.

Preferably, the microcapsule wall material is water glass and slag, and the slag is selected from one or more of carbide slag, desulfurized gypsum and coal powder ash.

A preparation method of the microcapsule of sustainable self-healing coal mine ventilation sealing material is carried out in the following steps.

(1) weighing an amount of the bacterial lyophilized powder and the substrate respectively, and adding the weighed bacterial lyophilized powder into a proper amount of water to enable homogeneous dispersion, and then adding the weighed and uniformly-mixed substrate into the homogenously-dispersed bacterial lyophilized powder to perform uniform mixing into a agglomerate mixture, and kneading the agglomerated microcapsule core material to be non-sticky to hand;

(2) adding the above agglomerate mixture slowly into a multifunctional pelletizer at the speed of 50 g/times to perform cake pressing, extrusion and pelletization, performing rounding drying at the temperature of 25-30° C., and then performing screening to obtain microcapsule core material micro-granules of single granule size;

(3) weighing the wall material according to the mass ratio, 1:1.2-1.5, of the core material micro-granule to the wall material, and uniformly mixing the powdered wall material;

(4) spraying the uniformly-mixed microcapsule wall material to a surface of the obtained microcapsule core material micro-granule, performing rounding drying at the temperature of 25-30° C., and then uniformly spraying one layer of water glass and then drying so as to obtain microcapsule granules of single granule size.

Preferably, at step (1) the mass ratio of water to the substrate is 1:0.75-0.9.

The present disclosure has the following beneficial effects. Compared with the existing coal mine ventilation sealing material crack repairing method, the present disclosure has the following prominent advantages in the actual applications:

1) The microbial self-healing system can realize automatic detection and automatic healing of cracks.

2) Bacteria can perform several crack repairs and the healing performance is sustainable.

3) Calcium carbonate produced by bacteria mineralization is matched with the body material, thereby ensuring environmental protection and no pollution.

4) The microcapsule wall material is an inorganic material which has good compatibility with both bacteria and the cement-based material, so that the bacteria is well protected against the impact of the external environment, increasing the life of microorganisms and reducing the impact on the mechanical property of the cement-based material.

5) In a cement hydration stage, the carbide slag in the wall material provides sufficient OH⁻ capable of destroying a glass structure of the surface of the coal powder ash and releasing its internal active SiO₂ and Al₂O₃ which participate in hydration of cement together with the desulfurized gypsum to generate compact C—S—H and C-A-H gel, and the microcapsule doped with a given amount can increase the strength of the cement-based material.

6) Without additional culture medium, smart repair of material can be achieved.

7) In a case of occurrence of self-healing, only a small number of microbial spores that are affected by cracks are activated and other microbial spores are still in sleeping state, so that the self-healing system is effective for long while avoiding the impact of a large number of dead microbial spores on the strength of the ventilation sealing body material.

8) The self-healing system is capable of realizing large-scope and uniform repair because a large number of microbial capsules are uniformly distributed in the ventilation sealing matrix material.

In summary, by use of urease-producing bacteria, there is provided a method of protecting bacteria to survive a long time in the cement-based material, supplying sufficient nutrient substances and reducing the impact of the doping of bacteria on the mechanical property of the cement-based material. In this method, the bacterial lyophilized powder and its substrate are prepared into microcapsules which are added into the cement-based material when the cement-based material is mixed. In this case, once concrete cracks, the microcapsules breaks and the spores in the material are activated to perform normal metabolism so as to induce precipitation of calcium carbonate continuously, thereby continuously realizing self-healing of coal mine ventilation sealing material cracks. The preparation method of the present disclosure is simple in operation and the microcapsules can be mass-produced.

### BRIEF DESCRIPTION OF THE DRAWINGS

FIG. 1 is a schematic diagram of a process of preparation of microcapsules.

FIG. 2 is a schematic diagram of a specimen prepared by adding microcapsules into a cement-based material.

In FIG. 1, a: agglomeration, b: cake pressing, c: pelletization, d: formed microcapsules.

### DETAILED DESCRIPTION OF THE EMBODIMENTS

The present disclosure will be further detailed in combination with the following examples.

### EXAMPLE 1

A preparation method of a microcapsule of sustainable self-healing coal mine ventilation sealing material crack is provided in this example. Bacterial lyophilized powder in a microcapsule core material in the example is sporosarcina pasteurii, a substrate is urea, calcium chloride, yeast powder, hydroxy propyl methyl cellulose and microcrystalline cellulose, and a microcapsule wall material is carbide slag, desulfurized gypsum and water glass.

1. 30 g of yeast powder, 8 g of hydroxy propyl methyl cellulose, 150 g of microcrystalline cellulose were accurately weighed and uniformly mixed, 3.5 g of sporosarcina pasteurii already weighed was added into 240 g of water to enable homogeneous dispersion, and then 75 g of calcium chloride and urea already weighed respectively was dissolved in the water in which sporosarcina pasteurii was dispersed, and then the liquid solution was added into uniformly-mixed yeast powder, hydroxy propyl methyl cellulose and microcrystalline cellulose and mixed uniformly to agglomeration, and then the agglomerated microcapsule core material was kneaded to be non-sticky to hand so as to prepare a microcapsule core material.

2. The agglomerate mixture was added slowly into a multifunctional pelletizer at the speed of 50 g/times to perform cake pressing, extrusion and pelletization, the prepared micro-granules of the microcapsule core material were dried in a rounder at the temperature of 28° C. to obtain microcapsule core material micro-granules of single granule size after screening.

3. A given amount of microcapsule wall material was weighed according to the mass ratio, 1:1.40, of the microcapsule core material to the microcapsule wall material, where the mass ratio of the carbide slag, desulfurized gypsum and water glass in the microcapsule wall material is 1:1.3:0.8; after the above materials were accurately weighed, the carbide slag and the desulfurized gypsum were added into the water glass and uniformly mixed.

4. The surface of the microcapsule core material micro-granule was uniformly sprayed with the above mixture, dried at the temperature of 28° C., then uniformly sprayed with a layer of water glass and then dried to form a layer of protective film on the surface of microcapsule, thereby preparing a microcapsule granule of single granule size.

### EXAMPLE 2

A preparation method of a microcapsule of sustainable self-healing coal mine ventilation sealing material crack is provided in this example. Bacterial lyophilized powder in a microcapsule core material in the example is bacillus cereus, a substrate is urea, calcium lactate, peptone, yeast powder, xanthan gum, and microcrystalline cellulose, and a microcapsule wall material is coal powder ash, carbide slag, desulfurized gypsum and water glass.

1. 5 g of peptone, 25 g of yeast powder, 5 g of xanthan gum, and 150 g of microcrystalline cellulose were accurately weighed and uniformly mixed, 3 g of bacillus cereus already weighed was added into 225 g of water to enable homogeneous dispersion, and then 75 g of calcium lactate and urea already weighed respectively was dissolved in the water in which bacillus cereus was dispersed, and then the liquid solution was added into uniformly-mixed peptone, yeast powder, xanthan gum and microcrystalline cellulose and mixed uniformly to agglomeration, and then the agglomerated microcapsule core material was kneaded to be non-sticky to hand so as to prepare a microcapsule core material.

2. The agglomerate mixture was added slowly into a multifunctional pelletizer at the speed of 50 g/times to perform cake pressing, extrusion and pelletization, the prepared micro-granules of the microcapsule core material were dried in a rounder at the temperature of 20° C. to obtain microcapsule core material micro-granules of single granule size after screening.

3. A given amount of microcapsule wall material was weighed according to the mass ratio, 1:1.30, of the microcapsule core material to the microcapsule wall material, where the mass ratio of the carbide slag, desulfurized gypsum, coal powder ash and water glass in the microcapsule wall material was 1:1.25:0.4:0.9; after the above materials were accurately weighed, the carbide slag, coal powder ash and the desulfurized gypsum were added into the water glass and uniformly mixed. The surface of the microcapsule core material micro-granule was uniformly sprayed with the above mixture, dried at the temperature of 20° C., then uniformly sprayed with a layer of water glass and then dried to form a layer of protective film on the surface of microcapsule, thereby preparing a microcapsule granule of single granule size.

### EXAMPLE 3

A preparation method of a microcapsule of sustainable self-healing coal mine ventilation sealing material crack is provided in this example. Bacterial lyophilized powder in a microcapsule core material in the example is bacillus sphaericus, a substrate is urea, calcium formate, peptone, yeast powder, xanthan gum, and microcrystalline cellulose, and a microcapsule wall material is coal powder ash, carbide slag, desulfurized gypsum and water glass.

1. 5.5 g of peptone, 28 g of yeast powder, 5.5 g of xanthan gum, and 150 g of microcrystalline cellulose were accurately weighed and uniformly mixed, 3.3 g of bacillus sphaericus already weighed was added into 240 g of water to enable homogeneous dispersion, and then 75 g of calcium formate and urea already weighed respectively was dissolved in the water in which bacillus sphaericus was dispersed, and then the liquid solution was added into uniformly-mixed peptone, yeast powder, xanthan gum and microcrystalline cellulose and mixed uniformly to agglomeration, and then the agglomerated microcapsule core material was kneaded to be non-sticky to hand so as to prepare a microcapsule core material.

2. The agglomerate mixture was added slowly into a multifunctional pelletizer at the speed of 50 g/times to perform cake pressing, extrusion and pelletization, the prepared micro-granules of the microcapsule core material were dried in a rounder at the temperature of 20° C. to obtain microcapsule core material micro-granules of single granule size after screening.

3. A given amount of microcapsule wall material was weighed according to the mass ratio, 1:1.25, of the microcapsule core material to the microcapsule wall material, where the mass ratio of the carbide slag, coal powder ash, and water glass in the microcapsule wall material was 1:0.4:0.9; after the above materials were accurately weighed, the carbide slag, and coal powder ash were added into the water glass and uniformly mixed. The surface of the microcapsule core material micro-granule was uniformly sprayed with the above mixture, dried at the temperature of 20° C., then uniformly sprayed with a layer of water glass and then dried to form a layer of protective film on the surface of microcapsule, thereby preparing a microcapsule granule of single granule size.

A cuboid specimen of 40×40×160 mm is prepared by adding the microcapsule prepared in the example 2 into the cement-based material. The doping amount of the microcapsule is 0%, 3%, 5%, 7%, 9%, 11% and 13% of the cement mass respectively, where the ratio of water to cement is 0.65, the ratio of gel to sand is 1:3. After being subjected to standard curing for 14d, the specimen undergoes flexural and compressive tests to indicate the impacts of different microcapsule doping amounts on the mechanical property of the cement-based material, with test results shown in Table 1.

TABLE 1
Strengths of cement specimen 14 d of
different microcapsule doping amounts

| | Microcapsule doping amount/wt % | | | | | | |
|---|---|---|---|---|---|---|---|
| | 0% | 3% | 5% | 7% | 9% | 11% | 13% |
| Flexural | 4.63 | 4.83 | 5.04 | 4.14 | 3.64 | 3.16 | 2.99 |
| strength/MPa | | | | | | | |
| Compressive | 11.99 | 12.03 | 12.29 | 7.54 | 6.91 | 6.63 | 5.36 |
| strength/MPa | | | | | | | |

It can be seen from the Table 1 that when the microcapsule doping amount is 3% and 5%, the specimen strength can be increased, and when the microcapsule doping amount exceeds 5%, the specimen strength gradually decreases. The reason for the above is as follows: in the cement hydration stage, the carbide slag in the wall material provides sufficient OH⁻ capable of destroying a glass structure of the surface of the coal powder ash and releasing its internal active SiO₂ and Al₂O₃ which participate in hydration of cement together with the desulfurized gypsum to generate compact C—S—H and C-A-H gel, the microcapsule doped with a given amount can increase the strength of the cement-based material, and along with increase of the microcapsule doping amount, the compactness of the cement-based material in the specimen is decreased, resulting in a lower strength.

Of course, the above descriptions are not intended to limit the present disclosure, and the present disclosure is not limited to these above examples. Changes, modifications, addition or substitutions made by those skilled in the prior art within the essence scope of the present disclosure shall fall within the scope of protection of the present disclosure.

1. A microcapsule of sustainable self-healing coal mine ventilation sealing material crack, comprising a microcapsule core material and a microcapsule wall material, wherein the microcapsule core material is prepared using a bacterial lyophilized powder and a substrate.

2. The microcapsule of sustainable self-healing coal mine ventilation sealing material crack according to claim 1, wherein a mass ratio of the microcapsule core material to the microcapsule wall material is 1:1.2-1.5.

3. The microcapsule of sustainable self-healing coal mine ventilation sealing material crack according to claim 2, wherein a granule size of the microcapsule core material is 1.9-2.5 mm with a wall thickness being 60-100 μm.

4. The microcapsule of sustainable self-healing coal mine ventilation sealing material crack according to claim 3, wherein the bacterial lyophilized powder in the microcapsule core material is selected from one or more of bacillus sphaericus, sporosarcina pasteurii and bacillus cereus.

5. The microcapsule of sustainable self-healing coal mine ventilation sealing material crack according to claim 3, wherein the substrate in the microcapsule core material is urea, soluble calcium salt, nutrient substance and thickener; the soluble calcium salt is selected from one or more of calcium chloride, calcium lactate, calcium nitrate, calcium formate and calcium acetate, the nutrient substance is selected from one or more of inosine, peptone and yeast powder; and the thickener is selected from one or more of microcrystalline cellulose, xanthan gum, and hydroxy propyl methyl cellulose.

6. The microcapsule of sustainable self-healing coal mine ventilation sealing material crack according to claim 1, wherein a mass ratio of the bacterial lyophilized powder to the substrate is 1:50-70.

7. The microcapsule of sustainable self-healing coal mine ventilation sealing material crack according to claim 2, wherein the microcapsule wall material is water glass or slag, and the slag is selected from one or more of carbide slag, desulfurized gypsum and coal powder ash.

8. A preparation method of the microcapsule of sustainable self-healing coal mine ventilation sealing material crack according to claim 1, comprising the following steps:
(1) weighing an amount of the bacterial lyophilized powder and the substrate respectively, and adding the weighed bacterial lyophilized powder into a proper amount of water to enable homogeneous dispersion, and then adding the weighed and uniformly-mixed substrate into the homogenously-dispersed bacterial lyophilized powder to perform uniform mixing into a agglomerate mixture, and kneading the agglomerated microcapsule core material to be non-sticky to hand;
(2) adding the above agglomerate mixture slowly into a multifunctional pelletizer at the speed of 50 g/times to perform cake pressing, extrusion and pelletization, performing rounding drying at the temperature of 25-30° C., and then performing screening to obtain microcapsule core material micro-granules of single granule size;
(3) weighing the wall material according to the mass ratio, 1:1.2-1.5, of the core material micro-granule to the wall material, and uniformly mixing the powdered wall material;
(4) spraying the uniformly-mixed microcapsule wall material to a surface of the obtained microcapsule core material micro-granule, performing rounding drying at the temperature of 25-30° C., and then uniformly spraying one layer of water glass and then drying so as to obtain microcapsule granules of single granule size.

9. The preparation method of the microcapsule of sustainable self-healing coal mine ventilation sealing material crack according to claim 8, wherein at step 1, the mass ratio of water to the substrate is 1:0.75-0.9.
